(19) United States
(12) Patent Application Publication
(10) Pub. No.: US 2017/0198028 A1
(43) Pub. Date: Jul. 13, 2017

(54) CRYSTALLIZATION METHODS FOR PURIFICATION OF MONOCLONAL ANTIBODIES

(71) Applicant: NOVARTIS AG, Basel (CH)

(72) Inventors: Dariusch Hekmat, Munich (DE); Bernhard Helk, Loerrach (DE); Henk Schulz, Freiburg im Breisgau (DE); Benjamin Smejkal, Wolfersdorf (DE)

(21) Appl. No.: 15/429,514

(22) Filed: Feb. 10, 2017

Related U.S. Application Data
(62) Division of application No. 14/400,179, filed on Nov. 10, 2014.
(60) Provisional application No. 61/645,855, filed on May 11, 2012.

(51) Int. Cl. C07K 16/00; C07K 1/30

(52) U.S. Cl. CPC C07K 16/00; C07K 1/306

(57) ABSTRACT

This disclosure relates to methods for crystallization of antibodies from cell-free culture supernatant.

### FIELD OF THE DISCLOSURE

This disclosure relates to methods for crystallizing and purifying monoclonal antibodies.

### BACKGROUND OF THE DISCLOSURE

This disclosure relates to high yield preparation and purification of monoclonal antibodies in crystal form directly from culture supernatant (e.g., cell-free supernatant of a cell culture that secretes monoclonal antibody into the supernatant). Problems with crystallization of proteins include, for example: 1) the need for specialized equipment; 2) production of polymorphous crystals; 3) the need for seeding to initiate crystallization; 4) time-intensive processes (e.g., 60-80 hours); 5) chromatography steps prior to crystallization (e.g., protein A, ion exchange (IEX); 7) the use of unfavorable additives and/or excipients (e.g., polyethylene glycol); and 8) storage difficulties. While monoclonal antibodies have been previously crystallized directly from cell culture supernatant, the yield was low. In addition, prior methods required the use of an additive such as polyethylene glycol. The methods described herein surprisingly provide for the production of high purity, crystallized monoclonal antibodies in high yield from cell-free culture supernatant without use of costly steps or equipment. These new methods provide many advantages including, for example, a highly concentrated, stable crystallized antibody suitable for formulation into pharmaceutical products as well as significant time and cost benefits.

### BRIEF DESCRIPTION OF THE DRAWINGS

FIG. 1. Exemplary crystallization methods.

FIG. 2A, FIG. 2B, and FIG. 2C. Exemplary crystallization conditions.

FIG. 3A and FIG. 3B. Exemplary crystals.

FIG. 4. Effects of pH on crystallization under exemplary conditions.

FIG. 5. Kinetics of crystallization under exemplary conditions.

FIG. 6. Kinetics of crystallization under additional exemplary conditions

FIG. 7A, FIG. 7B, FIG. 7C, FIG. 7D, and FIG. 7E. Exemplary crystals.

FIG. 8A, FIG. 8B, and FIG. 8C. Exemplary crystals.

FIG. 9. Exemplary crystals.

FIG. 10. Exemplary crystals.

FIG. 11. Exemplary crystals.

### SUMMARY OF THE DISCLOSURE

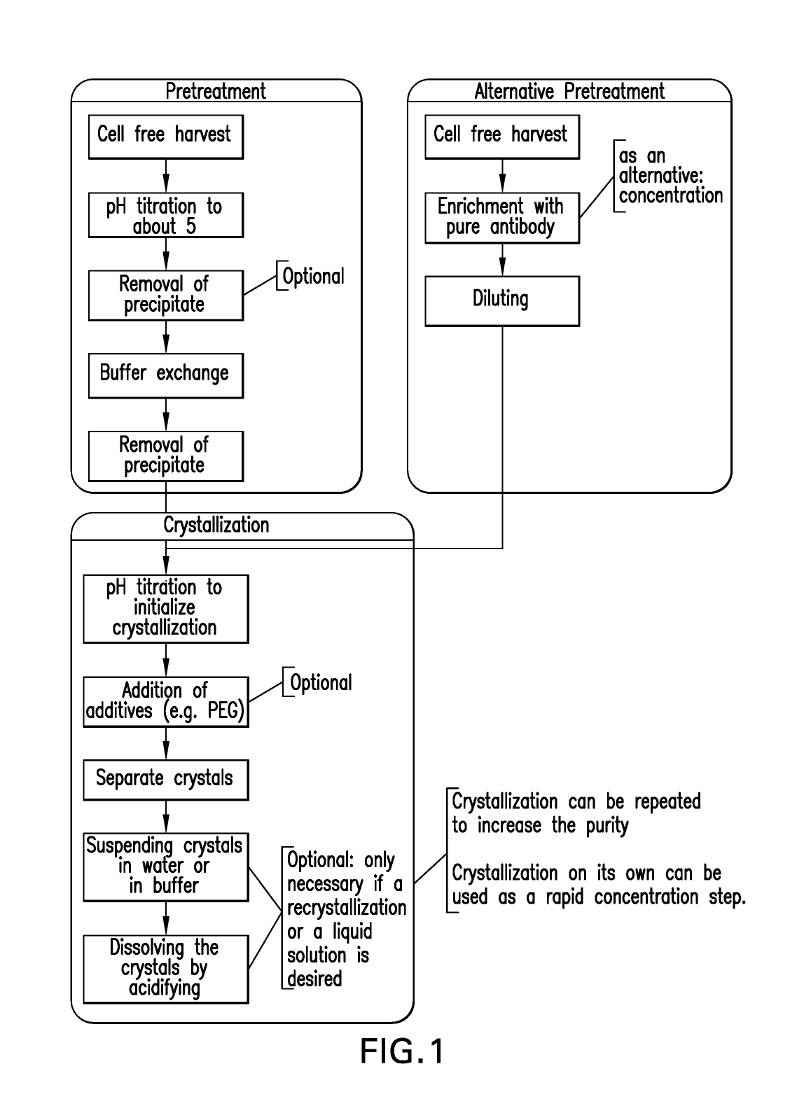

This disclosure relates to inventive methods that solve problems typically encountered during the purification of monoclonal antibodies. The methods described herein are surprisingly useful for providing purified monoclonal antibody preparations from mixtures comprising monoclonal antibodies. In some embodiments, the inventive methods described herein provide for the production of high purity, crystallized monoclonal antibodies in high yield directly from cell-free culture supernatant. In particular embodiments described herein, this is accomplished using a low ionic strength buffer. In some embodiments, the methods for preparing monoclonal antibodies in crystal form may comprise introducing low ionic strength buffer into a cell-free cell culture supernatant containing monoclonal antibodies under appropriate pH conditions that promote precipitation. The resulting precipitate, containing mainly impurities, is then typically removed (e.g., to produce a clarified supernatant). The clarified supernatant may then be optionally concentrated. An appropriate buffer may then be introduced to produce a pretreated solution. The pH of the pretreated solution may then be at, or be adjusted to, an appropriate level at which the protein crystallizes (e.g., for a protein crystallizing at or near the pI of 6.8, the pH should be about 6.8). One or more additives (e.g., sodium chloride, polyethylene glycol, sugar) may also be included. The resultant crystals may then be isolated by, for example, centrifugation. Certain embodiments are illustrated in FIG. 1. Some embodiments provide a product comprising at least about 50%, 75%, 80%, 85%, 90%, 95%, or 99% of the protein (e.g., antibody) present in the initial cell-free culture supernatant. Prior to use, the crystals may be dissolved in an appropriate solution and then optionally re-crystallized by adjusting the pH of the solution to the range in which the protein crystallizes (e.g., for a protein crystallizing at or near the pI of 6.8, the pH should be about 6.8). The size of the resulting crystals may be controlled by, for example, adjusting the starting protein concentration of the cell culture supernatant and/or stirring the substrate of any step at a particular speed. Compositions containing crystallized antibodies, and re-dissolved antibodies are also provided.

The methods of the invention can be free of chromatography steps. An advantage of excluding chromatography from one or more steps of the inventive methods includes significant reduction of the time in producing purified monoclonal antibodies in crystal form. Particular embodiments of the invention include those wherein no chromatography is carried out on a starting material or a resultant product of a recited step. Particular embodiments of the invention include those wherein no chromatography is carried out prior to the crystallization step.

### DETAILED DESCRIPTION

As described briefly above and in more detail below, this disclosure relates to methods for purification of monoclonal antibodies. The methods described herein may be surprisingly used to provide purified monoclonal antibody preparations from compositions comprising monoclonal antibodies. As mentioned above, this has been accomplished using a low ionic strength buffer. In some embodiments, the methods described herein provide for the production of highly pure, crystallized monoclonal antibodies in high yield directly from cell-free culture supernatant. In some embodiments, the methods for preparing monoclonal antibodies in crystal form may include one or more of the steps of providing a cell-free cell culture supernatant comprising monoclonal antibodies, introducing (e.g., diluting or replacing (e.g., by partial or complete dialysis)) a low ionic strength buffer to the cell-free cell culture supernatant in an amount sufficient to promote the crystallization of said antibody, and adjusting the pH of the resultant solution to produce crystals, and isolating the crystals, wherein at least 50% of the antibody contained in the cell-free cell culture supernatant is isolated.

In some embodiments, the methods for preparing monoclonal antibodies in crystal form may include one or more of the steps of: determining the pH range in which the antibodies crystallize in a low ionic strength buffer, introducing (diluting or replacing (e.g., by partial or complete dialysis)) said buffer to the cell culture supernatant in an amount sufficient to promote the crystallization of said antibody in the pH range to produce a pre-crystallization solution, adjusting the pH of said pre-crystallization solution to the determined range in the above determining step to produce crystals, and isolating the crystals, wherein at least 50% of the antibody contained in the cell-free culture supernatant is isolated.

In some embodiments, methods for preparing monoclonal antibodies in crystal form may include one or more of the steps of: a) obtaining cell-free culture supernatant of a hybridoma producing a monoclonal antibody and optionally concentrating the same; b) dialyzing the supernatant against a buffer (e.g., a low ionic strength buffer) to provide an appropriate pH; c) removing precipitate formed in step b) from the supernatant, if present therein, to produce a clarified supernatant; d) optionally concentrating the clarified supernatant; e) optionally dialyzing the clarified supernatant of c) or d) against an appropriate buffer to produce a pretreated solution; f) removing precipitate from the pretreated solution of step e), if present therein; g) adjusting the pH of the pretreated solution of step e) or f) to an appropriate level at which the monoclonal antibody crystallizes (e.g., for a monoclonal antibody crystallizing at or near the pI of 6.8, the pH should be about 6.8) and optionally introducing one or more additives to produce a crystallization solution; and, h) isolating the crystals formed in step g) by, for example, centrifugation.

While monoclonal antibodies have been previously crystallized from cell culture supernatant, the yield was low. Previous methods typically require the use of an additive such as polyethylene glycol. And standard crystallization screens typically do not include low-ionic strength buffers. The influence of pH on the solubility of the protein is very high (which may decrease supersaturation potential). As shown herein (e.g., the Examples), a simple change of pH of a protein solution containing a low ionic strength buffer could surprisingly be used to reduce the solubility of a monoclonal antibody (e.g., from >200 g L⁻¹ at pH 5 to 0.3 g L⁻¹ at pH 6.8), in turn leading to very high supersaturation and crystallization (e.g., no precipitation at pH 6.8) with precipitation of impurities (some of which could inhibit crystallization) at pH 5 (at which antibody was soluble). The methods described herein unexpectedly provide for the production of high purity, crystallized monoclonal antibodies in high yield directly from cell-free culture supernatant. Certain embodiments are illustrated in FIG. 1. In certain embodiments, the clarified supernatant produced in step a) may be concentrated. In some embodiments, the pH may be adjusted using a buffer optionally comprising one or more additives selected from the group consisting of sodium chloride, polyethylene glycol, and a sugar. Some embodiments surprisingly provide a product comprising at least about 50%, 75%, 80%, 85%, 90%, 95%, or 99% of the antibody present in the initial cell-free culture supernatant of, for example, step a) above (e.g., a high yield). Prior to use, the crystals may be solubilized in an appropriate solution (e.g., a pharmaceutical composition). The crystals may also be dissolved and then optionally re-crystallized by, for example, adjusting the pH of the solution to an appropriate level (e.g., for monoclonal antibody having a pI of about 6.8, the pH should be about 6.8). In these methods, the size of the resulting crystals may be controlled by, for example, adjusting the starting protein concentration of the cell culture supernatant and/or stirring the substrate of any step at a particular speed. Additional details of these methods, the products produced thereby, and uses for such products, are explained below.

The methods described herein typically begin with a cell-free culture supernatant of a cell producing a monoclonal antibody to be crystallized (e.g., step a)). It should be understood that other starting materials (e.g., hybridoma culture, ascites, a semi-purified, or purified preparation containing the antibody to be crystallized) may also be used. These methods may also be suitable for isolation of “purified” polyclonal antibodies from sera and the like. Regarding a cell-free culture supernatant, it may be used straight (e.g., directly) from culture or concentrated prior to processing. The cell-free culture supertant may be concentrated by a factor of, for example, about 2, 3, 4, 5, 6, 7, 8, 9, 10, 11, 12, 13, 14, 15, 16, 17, 18, 19 or 20 to provide a lesser volume and, therefore, a higher concentration of proteins (and other components) (e.g., 100 ml to 10 ml being a factor of 10, or 10:1). The protein concentration of the cell-free supernatant may be, for example, about 1-100 g/L, such as about 10 g/L, 25 g/L, or 50 g/L. Concentration may be achieved using any of several widely available technique such as, for example, centrifugation, ammonium sulphate concentration, spin centrufugation and/or ultrafiltration (e.g., Amicon Ultra-15 Centrifugal Filter Unit with Ultracel-10 membrane), as would be understood by one of ordinary skill in the art. These and other suitable starting materials would be understood by one of ordinary skill in the art.

As described in the Examples, the cell-free culture supernatant (e.g., optionally concentrated) typically contains many components other than the monoclonal antibody (e.g., impurities). The cell culture media may not be appropriate for use with the methods described herein and may, therefore, be exchanged for another buffer. Thus, the cell-free culture supernatant may be exchanged for (e.g., diluted and/or dialyzed against) a buffer (e.g., a low ionic strength buffer such as a histidine buffer such as 10 mM histidine, 10 mM NaCl, adjusted to pH 5 using acetic acid using a crossflow ultrafiltration unit) containing components compatible with the methods described herein (e.g., to provide a suitable pH of about pH 4-10 (e.g., about 4.9, 5.0, 5.5, 6.0, 6.5, 6.8, 7.0, 7.5, 8.0, 8.5, 9.0 or 9.5)). The buffer may be, for example, a “low ionic strength” buffer (e.g., providing a conductivity of about 1, 2, 3, 4, 5, 6, 7, 8, 9, 10, 11 or 12 mS cm⁻¹, or lower). For instance, exemplary suitable buffers may include 10 mM histidine buffer with or without one or more salts such as 10 mM histidine buffer/20 mM sodium chloride or 10 mM histidine buffer/100 mM sodium chloride (conductivity: 10.9 mS cm⁻¹). Such buffers may also facilitate the precipitation of impurities from the cell-free culture supernatant. In some embodiments, a dialysis tubular membrane (Dialysis Tubing Visking (MWCO) 14000) may be utilized. Where impurities are precipitated during and/or following dialysis/buffer exchange, the precipitate may be separated from the antibodies (and other non-precipitated components) using a technique such as filtration or centrifugation (e.g., 3000-5000 rcf (e.g., 3200 rcf, 5252 rcf) for 10, 15 or 20 minutes). In some embodiments, the resultant solution, which contains antibodies, may be referred to as a “clarified supernatant” (or, as in the Examples, a “pre-treated harvest”). It is preferred that the conductivity of a clarified supernatant (or pre-treated harvest) be about 0.1, 0.2, 0.3, 0.4, 0.46, 0.5, 0.6, 0.7, 0.8, 0.9. 1.0, 1.1, 1.2, 1.3, 1.4, 1.5, 1.6, 1.7, 1.8, 1.9, 2.0, 3.0, 4.0, 5.0, 6.0, 7.0, 8.0, 9.0, 10.0, 11 (e.g., 10.9), or 12 mS cm⁻¹. Other methods of preparing a pre-treated harvest for processing using the methods described herein may also be suitable, as would be understood by one of ordinary skill in the art.

The clarified supertant may then optionally be concentrated using, for example, any of several widely available techniques (e.g., centrifugation, ammonium sulphate concentration, and/or ultrafiltation), as would be understood by one of ordinary skill in the art. The clarified supernatant (either unconcentrated or concentrated) may then be optionally dialzyed against (e.g., exchanged for) another buffer (e.g., a low ionic strength buffer) to produce a “pre-treated solution” (e.g., a histidine buffer such as 10 mM histidine, 10 mM NaCl, adjusted to pH 5 using acetic acid using a crossflow ultrafiltration unit). The buffer may contain, for example, a buffering component (e.g., about 1-15 mM histidine (e.g., 3, 10, 14 mM) (about pH 4-10 (e.g., about 4.9, 5.0, 5.5, 6.0, 6.5, 6.8, 7.0, 7.5, 8.0, 8.5, 9.0 or 9.5)), one or more salts (e.g., NaCl), and/or one or more sugars (e.g., trehalose). Introduction of such buffers will typically result in the formation of a precipitate containing impurities. The precipitate may then be separated from the antibodies (and other non-precipitated components) using a technique such as filtration or centrifugation (e.g., 3000-5000 rcf (e.g., 3200 rcf, 5252 rcf) for 10, 15 or 20 minutes) to produce a “clarified pre-treated solution”. It is preferred that the conductivity of a clarified pre-treated solution be about 0.1, 0.2, 0.3, 0.4, 0.46, 0.5, 0.6, 0.7, 0.8, 0.9. 1.0, 1.1, 1.2, 1.3, 1.4, 1.5, 1.6, 1.7, 1.8, 1.9, 2.0, 3.0, 4.0, 5.0, 6.0, 7.0, 8.0, 9.0, 10.0, 11 (e.g., 10.9), or 12 mS cm⁻¹. This clarified pre-treated solution is then typically used as the substrate for crystallization, although the pre-treated harvest may also be suitable. Other methods of preparing a pre-treated solution for crystallization may also be suitable, as would be understood by one of ordinary skill in the art.

The pH of the pre-treated solution is then typically adjusted to an appropriate level at which a particular protein will crystallize. Typically, the appropriate pH is that which matches the pI of the protein to be crystallized. For example, the pH should be about 6.8 for a protein having a p1 of about 6.8. The pH may be provided by an appropriate buffer comprising, for instance, TRIS (e.g., about 2-20 mM TRIS (e.g., TRIS-HCl) such as about 4, 6, 7, 8, 9, 12, 12.8, 14, 15, 16, 18 mM), histidine (e.g., about 5-20 mM histidine such as about 10 or about 14.25 mM), HEPES (e.g., about 5-20 mM HEPES such as about 10 mM), phosphate (e.g., about 5-20 mM phosphate such as about 10 mM), cacodylate (e.g., about 5-20 mM such as about about 10 mM)), optionally along with an acid or base (e.g., acetic acid, HCl, and/or NaOH from, for example, a 10% or 0.5M stock solution) to provide a suitable pH depending on the protein (e.g., typically about pH 4-10 for a protein having a corresponding pI of from about 4-10 (e.g., about 4.9, 5.0, 5.5, 5.5-7.7, 6.0, 6.4, 6.5, 6.6, 6.8, 7.0, 7.5, 7.6, 8.0, 8.5, 9.0 or 9.5) and, optionally, one or more additional additives (e.g., about 5-100 mM NaCl (e.g., about 10, 15, 20, 25, 30, 40, 50, 60, 70, or 80 mM; about 2-8% w/v PEG MME 2000; about 2-8% w/v PEG MME 5000; about 0.8-1.6 mM MgSO₄; about 5-11 mM mM KCl (e.g., about 5.4 mM or 10.8 mM); about 1-10 mM CaCl₂ (e.g., about 1.8, 3.6, or 10 mM), about 2 mM EDTA; about 10-20 mM Li₂SO₄; about 10-40 mM LiCl (e.g., about 10, 20, 40 or 40 mM LiCl); about 10-20 mM NH₄Cl; about 10 mM (NH₄)₂SO₄; polyethylene glycol (e.g., PEG 1500, PEG 3000, PEG 10000 (e.g., at about 1-20% v/v such as about 2-8% (e.g., PEG 10000), 4%, 4-8% (e.g., PEG 3000), or 6-10% (e.g., PEG 1500) v/v); one or more sugars (e.g., sucrose, trehalose (e.g., about 40-400 mM such as about 250 mM); glycerin (e.g., about 5-20% v/v); 2-propanol (e.g., about 1-20% v/v); 1,4-dioxan (about 1-20% v/v); hexylene glycol (e.g., about about 1-5% v/v); ethanol (e.g., about 1-25% v/v); and/or hexyleneglycol) to produce a crystallization solution. Crystals may then be allowed to form over an appropriate period of time (about 1-150 minutes, such as about 3, 35, 60 or 120 minutes) at an appropriate temperature (e.g., 10° C., 20° C., 25° C., or 30° C., preferably about 10° C.). The protein concentration is typically about 0.1-100 g/L (e.g., about 1, 2, 4, 10, 25, 26, 50 g/L). An appropriate crystallization solution typically contains one, some, or all such components and provides for (e.g., induces) crystallization without precipitation. This may occur with or without seeding the crystallization solution with pre-formed crystals prior to or during crystallization. These crystals so formed may then be isolated by, for example, filtration or centrifugation (e.g., about 60-55000×g (e.g., 5252×g or 50377×g) for about 1-10 (e.g., about 3 minutes). The size of the crystals ultimately obtained using these methods may be controlled, to at least some extent, by, for example, adjusting the starting protein concentration of the cell culture supernatant to an appropriate level (e.g. about 1, 3, 5, 10, 25, 30, 35, 40, 45 or 50 g/L) and/or stirring the substrate in any one or more steps using particular equipment and/or at a particular speed. For example, in some embodiments, it may be beneficial to utilize an impeller that provides gentle hydrodynamic conditions (e.g. a power input per volume of less than about 1 W kg⁻¹) and/or maintains the crystals in suspension such as an appropriate multi-bladed segment impeller (e.g., a three-bladed segment impeller) and/or stirring at about 50-300 rpm (e.g., about 100, 110, 120, 130, 140, 150, 160, 170, 180, 190, 200, 210, 220, 230, 240, 250, 260, 270, 280, 290, or 300 rpm). These embodiments may provide high supersaturation, resulting in increased nucleation and crystal growth rates.

Increased nucleation rates may also be achieved by stirring at a specific range of the maximum local energy dissipation (εₘₐₓ). A suitable εₘₐₓ range may be, for example, from about 0.009 W kg⁻¹ to about 1.3 W kg⁻¹ (e.g., about any of 0.009, 0.01, 0.025, 0.05, 0.075, 0.1, 0.2, 0.3, 0.4, 0.5, 0.6, 0.7, 0.8, 0.9, 1.0, 1.1, 1.2, 1.3; or about 300 rpm). An optimal range may be, for example, between about 0.1 to about 0.4 W kg⁻¹ (e.g., about 0.1, 0.2, 0.3, or 0.4 W kg⁻¹). A suitable range may be dependent upon the type of reactor being used and may be determined by measurement of the drop size distribution of a silicone oil/surfactant/water emulsion. The suitable range may be selected as the point at which the silicon oil droplet size is reduced until an equilibrium is reached. The resulting drop size distribution in this system is entirely due to the reactor-specific comminution process. Thus, there is a dependency between the drop size and the maximum intensity of the local hydrodynamic stress εₘₐₓ (=maximum local energy dissipation) and a higher εₘₐₓ produces smaller particles (Henzler, H. Particle Stress in Bioreactors, Adv. Biochem. Eng. 67: 59 (2000)). Use of a silicone oil (baysilon oil PK 20) with a low viscosity of 20 mm² s⁻¹ and a density of 0.98 g·cm⁻³ (at 25° C.), characterization allows experiments to be performed even at low stirring rates (e.g., between 30 rpm and 350 rpm). For example, nine volumes of an aqueous solution with 8% v/v Triton X-100 were carefully layered with one volume of Sudan IV stained silicone oil. After stirring the system for 24 h at 10° C., the equilibrium particle diameter (d_{50,3}) which is the medium oil drop diameter of the volume sum distribution as determined by image analysis (e.g., optically). The d_{50,3} values used for crystallization were between 300 μm and 2400 μm. Other proteins (e.g., lysozyme) may be crystallized with a faster nucleation rate (e.g., a d_{50,3} value of about 440 μm). For an estimation of the corresponding εₘₐₓ value, a one-liter reactor was additionally characterized as explained below. The mean power consumption c was measured using a torque sensor. The ratio εₘₐₓ/ε can be estimated by the following equation (e.g., Henzler, supra, equation 20):

$$\frac{{ɛ}_{max}}{ɛ} \approx \frac{a}{{( d / D )}^{2} \times {( h / d )}^{2 / 3} \times {z}^{0} .6 \times {( sin α )}^{1} .15 \times {z}_{I}^{2 / 3} \times {( H / D )}^{- 2 / 3}}$$

where d is the diameter of the impeller, D is the inner tank diameter, h is the vertical height of impeller blade, H is the fill height, z is the number of impeller blades, a is the blade inclination to the horizontal, and z_{I} is the number of impellers. For example, where d=0.06 m; D=0.12 m; h=0.04 m; H=0.12 m; z=3; α=45°; z₁=1; a was calculated to be 4. The εₘₐₓ values were estimated to be between 0.03 W kg⁻¹ and 1 W kg⁻¹. The d_{50,3} value of about 440 μm would correspond to an estimated εₘₐₓ value of 0.5 W kg⁻¹. It was found that εₘₐₓ can be used as a parameter for scaling of protein crystallization independent from reactor design and geometrical dimensions. The existence of an optimum εₘₐₓ value which leads to a shorter crystallization process makes this parameter even more relevant.

As described in the Examples, the maximum crystal length in a 6 ml stirred batch reactor at 200 rpm was 60 μm and the maximum crystal length at 120 rpm was 120 μm. Thus, a slower stirring speed may provide for the formation of longer crystals. Other embodiments would be understood by one of ordinary skill in the art.

Accordingly, crystal formation may be accomplished using any of the following exemplary crystallization solutions/conditions, among others: 6 mM TRIS with up to about 15 mM NaCl; 8 mM TRIS with about 10, 20 or 30 mM NaCl; 10 g/L (protein), 7 mM TRIS, 25 mM NaCl; 50 g/L (protein), 12.8 mM TRIS, 40 mM NaCl; 12 or 16 mM TRIS and 20 mM NaCl; 25.9 g/L (protein), 14.25 mM histidine, 9 mM TRIS, and 25 mM NaCl; 10 g/L (protein), 10 mM Hepes buffer, pH 7.5; 10 g/L (protein), 10 mM cacodylate buffer, pH 7; 10 g/L (protein), 10 mM phosphate buffer, pH 6.5; 25 g/L (protein), 10 mM phosphate buffer, pH 6.5; 25 g/L (protein), 10 mM TRIS/HCl buffer, pH 7.5; 50 g/L (protein), 10 mM TRIS/HCl buffer, pH 7.5; 2, 4, or 10 g/L (protein), 10 mM histidine, 10 mM TRIS, 10 mM NaCl, 5-20% glycerin; 2, 4, or 10 g/L (protein), 10 mM histidine, 10 mM TRIS, 10 mM NaCl, 1-20% 2-propanol; 2, 4, or 10 g/L (protein), 10 mM histidine, 10 mM TRIS, 10 mM NaCl, 1-20% 1,4-dioxan; 2, 4, or 10 g/L (protein), 10 mM histidine, 10 mM TRIS, 10 mM NaCl, 1-5% hexylene glycol; 2, 4, or 10 g/L (protein), 10 mM histidine, 10 mM TRIS, 10 mM NaCl, 1-22% ethanol; 1, 2, 4, or 10 g/L (protein), 10 mM histidine, 10 mM TRIS, 10 mM NaCl, 6-10% PEG 1500; 1, 2, 4, or 10 g/L (protein), 10 mM histidine, 10 mM TRIS, 10 mM NaCl, 4-8% PEG 3000; 1, 2, 4, or 10 g/L (protein), 10 mM histidine, 10 mM TRIS, 10 mM NaCl, 2-8% PEG 10000; or 25 g/L (protein), 52 mM trehalose, 10 mM histidine, 15 mM TRIS, pH 6.8. Preferred among these, but not intended to be limiting in any way, may include histidine as buffer; NaCl to adjust the ionic strength; NaOH, TRIS, acetic acid, or HCl to adjust the pH; PEG 10000 as additive; and trehalose to generate an isotonic solution. As described above, crystallization may be carried out at any appropriate pH (e.g., about 5.5 to about 7.7, preferably about 6.8), temperature (e.g., 0° C., 5° C., 10° C., 20° C., 25° C., or 30° C., preferably about 10° C.), and time (about 1-150 minutes, such as about 3, 35, 60 or 120 minutes). In some embodiments, equilibrium may be achieved at between 1-60 minutes (e.g., 90% in less than 3 or 30 minutes). It is also preferred that the yield of antibody from the cell-free culture supernatant is high, being greater than about 30% to about 100% (e.g., about 30%, 35%, 40%, 45%, 50%, 55%, 60%, 65%, 70%, 75%, 77%, 80%, 85%, 90%, 90.5%, 95%, 95.8%, 98.2%, or 99%). Surprisingly, the methods described herein provide such high yield directly from cell-free culture supernatant without requiring an initial purification of the monoclonal antibodies therein and/or the use of additives such as polyethylene glycol. Thus, in some embodiments, the methods described herein provide crystallized monoclonal antibodies in high yield directly from cell-free culture supernatant (e.g., without chromatographic purification) using a crystallization solution that does not include polyethylene glycol. The crystallized antibodies typically provide acceptable long-term storage characteristics (e.g., low aggregation and fragments). For example, after removing any liquid by centrifugation, the crystals should exhibit low aggregate and fragment formation (e.g., less than about 1% and 2%, respectively (e.g., about 0.5% aggregates and about 1.5% fragments)).

The crystallized monoclonal antibodies produced using the processes described herein may be formulated into compositions, some of which may be pharmaceutical compositions. Such compositions described herein may take any form suitable for use in research and/or administration to a host (e.g., a mammal such as a human being). Suitable forms include, for example, liquids, capsules, emulsions, granules, films, implants, liquid solutions, lozenges, multi-particulates, sachets, solids, tablets, troches, pellets, powders, and/or suspensions. Liquid formulations may include diluents, such as water and alcohols, for example, ethanol, benzyl alcohol, and the polyethylene alcohols, either with or without the addition of a pharmaceutically acceptable surfactant. Capsule forms may formed of gelatin (e.g., hard- or soft-shelled). Any of such compositions may include, for example, surfactants, lubricants, and inert fillers, such as lactose, sucrose, calcium phosphate, corn starch, and/or the like. Tablet forms may include, for example, excipients and/or other agents such as lactose, sucrose, mannitol, corn starch, potato starch, alginic acid, microcrystalline cellulose, acacia, gelatin, guar gum, colloidal silicon dioxide, disintegrants (e.g., croscarmellose sodium), talc, magnesium stearate, calcium stearate, zinc stearate, stearic acid, colorants, diluents, buffering agents, disintegrating agents, moistening agents, preservatives, and/or flavoring agents. Lozenges forms may also be used, typically with with an inert base, such as gelatin and glycerin, or sucrose and acacia, emulsions, gels, and the like. The compositions may also prepared in lyophilized form. Other forms may also be suitable, as would be understood by one of skill in the art.

Pharmaceutical compositions may take any of the forms described above, or as may be known in the art. Pharmaceutical compositions may be prepared using one or more pharmaceutically acceptable carriers prior to use in research and/or administration to a host (e.g., an animal such as a human being). A pharmaceutically acceptable carrier is a material that is not biologically or otherwise undesirable, e.g., the material may be used in research and/or administered to a subject, without causing any undesirable biological effects or interacting in a deleterious manner with any of the other components of the pharmaceutical composition in which it is contained and/or reaction in which the same is used. The carrier would naturally be selected to minimize any degradation of the active agent and to minimize any adverse side effects in the subject, as would be well known to one of skill in the art. Suitable pharmaceutical carriers and their formulations are described in, for example, Remington's: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally from about 5 to about 8 or from about 7 to about 7.5. Other carriers include sustained-release preparations such as semipermeable matrices of solid hydrophobic polymers containing polypeptides or fragments thereof. Matrices may be in the form of shaped articles, e.g., films, liposomes or microparticles. It will be apparent to those of skill in the art that certain carriers may be more preferable depending upon, for instance, the route of administration and concentration of composition being administered. Also provided are methods for treating disease by administering the composition (e.g., as a pharmaceutical composition) to a host in need of treatment. Suitable routes of administration include, for example, oral, buccal, rectal, transmucosal, topical, transdermal, intradermal, intestinal, and/or parenteral routes. Other routes of administration and/or forms of the compositions described herein may also be suitable as would be understood by those of skill in the art.

The compositions described herein may be used to treat various diseases, including but not limited to cancer and non-cancer conditions. The monoclonal antibodies produced as described herein, and/or compositions comprising the same, may be used in research to detect proteins and/or nucleic acid function/expression in cells, tissues, and the like in vivo and/or in vitro. For example, the monoclonal antibodies may be used to stain cells to identify those expressing a particular protein. The monoclonal antibodies may also be conjugated to a detectable label and/or cytotoxic moiety. Other uses for the monoclonal antibodies produced as described herein are also contemplated as would be readily ascertainable by one of ordinary skill in the art.

Kits comprising the reagents required to crystallize a monoclonal antibody from a cell-free supernatant are also provided. An exemplary kit may contain one or more crystallization solutions and/or buffers (e.g., for dialysis/buffer exchange). The kit may also include various types of equipment (e.g., filters or the like) that may be necessary to carry out the methods described herein. The kit may also include positive and/or negative controls that may be used to confirm the method is functioning as desired. Instructions for use may also be included. Kits comprising the monoclonal antibodies and/or compositions comprising the same are also provided. In some embodiments, the kits comprise one or more containers comprising a composition described herein, or mixtures thereof, and instructions for in vitro or in vivo use. For example, the kit may include a container comprising a composition described herein and instructions for introducing the same to a cell in vitro, such as by adding the composition to a cell culture in bulk or to single cells. Regarding in vivo use, a kit may include a container containing a composition of an antibody and instructions for administering the same to an animal (such as a human being) to prevent or treat a disease condition. Other embodiments of kits are also provided as would be understood by one of ordinary skill in the art.

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

It must be noted that, as used in the specification and the appended claims, the singular forms “a”, “an”, and “the” include plural referents unless the context clearly dictates otherwise. Thus, for example, reference to a fragment may include mixtures of fragments and reference to a pharmaceutical carrier or adjuvant may include mixtures of two or more such carriers or adjuvants. The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto. As used herein, a subject or a host is meant to be an individual. Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

All references cited herein are hereby incorporated in their entirety by reference into this disclosure. A better understanding of the present invention and of its many advantages will be had from the following examples, given by way of illustration.

### EXAMPLES

### Example 1

A. Protein, Salt and Buffer Concentration

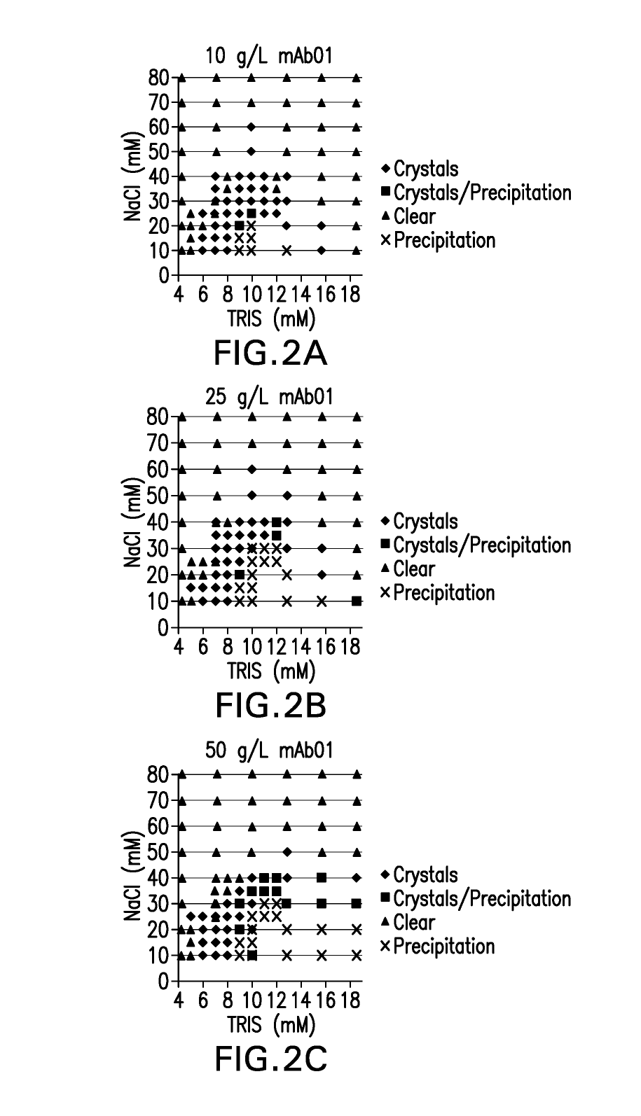

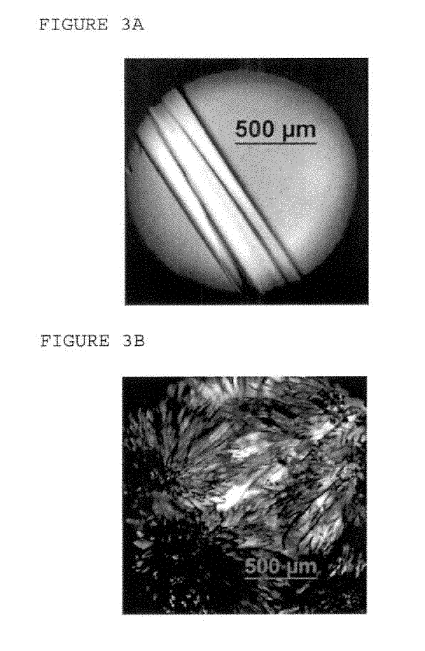

The crystallization region of pure mAb031 in 14 mM histidine buffer, pH 4.9, was determined in ml batch experiments (10 μl; Terasaki plates) at 10° C. by varying the protein concentration (10 g/L, 25 g/L, and 50 g/L), salt concentration (10, 20, 30, 40, 50, 60, 70, or 80 mM NaCl), and pH using various amounts of TRIS (4 mM=pH 5.5; 8 mM=pH 6.4; 9 mM=pH 6.6; 16 mM=pH 7.5; 18 mM=pH 7.6). As shown in FIGS. 2A-C, the conditions resulting in crystallization were clearly differentiated from those resulting in precipitation. For example, for each protein concentration tested, 6 mM TRIS and up to about 15 mM NaCl, or 8 mM TRIS and about 10, 20, or 30 mM NaCl resulted in crystal formation without precipitation. At 10 g/L, suitable conditions for crystallization were determined to also include, for example, 7 mM TRIS/25 mM NaCl (FIG. 3A). At 50 g/L, suitable conditions for crystallization were determined to also include, for example, 12.8 mM TRIS/40 mM NaCl (FIG. 3B). Other conditions resulting in crystallization are also apparent from FIGS. 2A-C.

B. Initiation of Crystallization by pH Adjustment

Pure mAb01 was crystallized in a 6 ml stirred batch experiment at 10° C. and 40 rpm. Crystallization conditions were 25.9 g/L mAb01, 14.25 mM histidine, 9 mM TRIS, and 25 mM NaCl. Crystallization was initiated by adjusting the pH to 6.6 using TRIS. A yield of 90.5% was reached after 35 minutes. At equilibrium (0.46 g/L mAb01), a yield of 98.2% was observed, with about 90% of the equilibrium being reached after about 30 minutes.

C. Other Buffer Systems/Additives/Salts

As shown above, the histidine/TRIS buffer system is very effective. Other buffer systems were also found to perform well. For example, crystallization with consistent crystal morphology was achieved using PEG 1500, PEG 3000, PEG 10000, glycerin, 2-propanol, 1,4-dioxan, hexyleneglycol, or ethanol. Several successfully tested systems are included: 10 g/L mAb01, 10 mM Hepes buffer, pH 7.5; 10 g/L mAb01, 10 mM cacodylate buffer, pH 7; 10 g/L mAb01, 10 mM phosphate buffer, pH 6.5; 25 g/L mAb01, 10 mM phosphate buffer, pH 6.5; 25 g/L mAb01, 10 mM TRIS/HCl buffer, pH 7.5; 50 g/L mAb01, 10 mM TRIS/HCl buffer, pH 7.5; 2, 4, or 10 g/L mAb01, 10 mM histidine, 10 mM TRIS, 10 mM NaCl, 5-20% glycerin; 2, 4, or 10 g/L mAb01, 10 mM histidine, 10 mM TRIS, 10 mM NaCl, 1-20% 2-propanol; 2, 4, or 10 g/L mAb01, 10 mM histidine, 10 mM TRIS, 10 mM NaCl, 1-20% 1,4-dioxan; 2, 4, or 10 g/L mAb01, 10 mM histidine, 10 mM TRIS, 10 mM NaCl, 1-5% hexylene glycol; 2, 4, or 10 g/L mAb01, 10 mM histidine, 10 mM TRIS, 10 mM NaCl, 1-22% ethanol; 1, 2, 4, or 10 g/L mAb01, 10 mM histidine, 10 mM TRIS, 10 mM NaCl, 6-10% PEG 1500; 1, 2, 4, or 10 g/L mAb01, 10 mM histidine, 10 mM TRIS, 10 mM NaCl, 4-8% PEG 3000; and, 1, 2, 4, or 10 g/L mAb01, 10 mM histidine, 10 mM TRIS, 10 mM NaCl, 2-8% PEG 10000. 10 g/L mAb01, 10 mM TRIS, 14 mM histidine, 10 mM CaCl₂; 10 g/L mAb01, 10 mM TRIS, 14 mM histidine, 10, or 20 mM Li₂SO₄; 2, 4, or 10 g/L mAb01, 10 mM TRIS, 14 mM histidine, 10, or 20 mM LiCl; 4, or 10 g/L mAb01, 10 mM TRIS, 14 mM histidine, 30 mM LiCl; 10 g/L mAb01, 10 mM TRIS, 14 mM histidine, 40 mM LiCl; 2, 4, or 10 g/L mAb01, 10 mM TRIS, 14 mM histidine, 10 mM NH₄Cl; 10 g/L mAb01, 10 mM TRIS, 14 mM histidine, 20 mM NH₄Cl; 4, or 10 g/L mAb01, 10 mM TRIS, 14 mM histidine, 10 mM (NH₄)₂SO₄; 2, 4, or 10 g/L mAb01, 13 mM TRIS, 10 mM histidine, 20 mM NaCl, 0.8 mM MgSO₄; 4, or 10 g/L mAb01, 13 mM TRIS, 10 mM histidine, 20 mM NaCl, 1.6 mM MgSO₄; 4, or 10 g/L mAb01, 13 mM TRIS, 10 mM histidine, 20 mM NaCl, 0.8 mM MgSO₄, 2 mM EDTA; 2, 4, or 10 g/L mAb01, 13 mM TRIS, 10 mM histidine, 20 mM NaCl, 1.6 mM MgSO₄, 2 mM EDTA; 4, or 10 g/L mAb01, 13 mM TRIS, 10 mM histidine, 20 mM NaCl, 1.8 mM CaCl₂; 10 g/L mAb01, 13 mM TRIS, 10 mM histidine, 20 mM NaCl, 3.6 mM CaCl₂; 2, 4, or 10 g/L mAb01, 13 mM TRIS, 10 mM histidine, 20 mM NaCl, 1.8 mM CaCl₂, 2 mM EDTA; 4 g/L mAb01, 13 mM TRIS, 10 mM histidine, 20 mM NaCl, 3.6 mM CaCl₂, 2 mM EDTA; 4, or 10 g/L mAb01, 13 mM TRIS, 10 mM histidine, 20 mM NaCl, 5.4 mM KCl; 2, or 10 g/L mAb01, 13 mM TRIS, 10 mM histidine, 20 mM NaCl, 10.8 mM KCl; 2, 4, or 10 g/L mAb01, 13 mM TRIS, 10 mM histidine, 20 mM NaCl, 5.4 mM KCl, 2 mM EDTA; 2, 4, or 10 g/L mAb01, 13 mM TRIS, 10 mM histidine, 20 mM NaCl, 10.8 mM KCl, 2 mM EDTA. Each of these sets of conditions provided acceptable results.

D. Effect of Temperature and pH on Crystal Stability

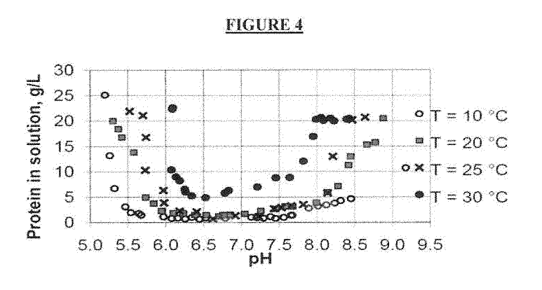

A mAb01 crystal suspension was produced in a 6 ml stirred batch at 10° C., 250 rpm (25 g/L mAb01, 20 mM NaCl, 10 mM histidine buffer (pH 5), 16 mM TRIS (final pH: 6.8). After reaching crystallization equilibrium, the temperature and pH were adjusted to 10° C., 20° C., 25° C., or 30° C. and the pH to 5.0, 5.5, 6.0, 6.5, 7.0, 7.5, 8.0, 8.5, 9.0 and 9.5, and stability measured as the amount of protein in solution (e.g., a higher amount of protein in solution indicates less stability). The results are shown in FIG. 4. As shown therein, lower temperatures provided a broader region of pH stability (e.g., at 10° C., stability was observed from about pH 5.5 to 7.7 with less stability at higher temperatures).

E. Dissolution of Pure mAb01 Crystals

Dissolution of pure mAb01 crystals was determined to occur quickly. Crystallization did not lead to aggregates and the biological activity of the crystallized antibodies was high. Dissolution of pure mAb01 was achieved within minutes by lowering the pH. Briefly, about 300 mg mAb01 crystals were suspended in 6 mL water and stirred at 20° C. in a 6 mL batch reactor. To dissolve the crystals, 4.5 mM acetic acid was added to adjust the pH to 5.4. The crystals dissolved within 5 minutes, producing a solution containing 35 g/L mAb01. In another experiment, mAb01 crystals obtained from a 1 L batch process were dissolved in 10 mM histidine buffer (pH 5) and adjusting the pH to 5 using 10% acetic acid. A highly concentrated liquid, viscous mAb01 solution of 200 g/L was obtained. In another test, a mAb01 preparation (8 g/L) was crystallized in 10 mM histidine/20-22 mM TRIS (pH 6.7) in a 5 mL stirred batch at 10° C. and separated by centrifugation (16100 rcf, 3 min, 10° C.). These crystals were resuspended and dissolved by adding 1 mL 10 mM histidine buffer (pH 4.9; pH was adjusted with 10% acetic acid) and pipetting at room temperature. The crystals dissolved within two minutes. Analysis (SEC) of the crystals showed no increase of byproducts or degradation products after crystallization. The biological activity after crystallization was increased slightly by about 4-5%.

F. Scale-Up of Pure mAb01 Crystallization from 6 mL Stirred Batch to a 1 L Stirred Batch Reactor with Extremely Fast Crystallization Kinetics and High Yields

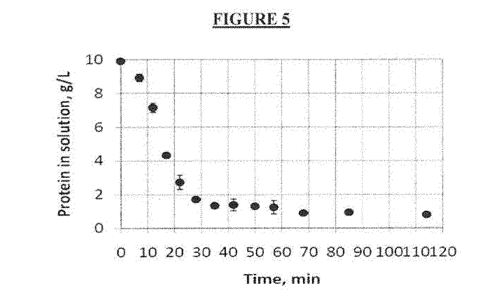

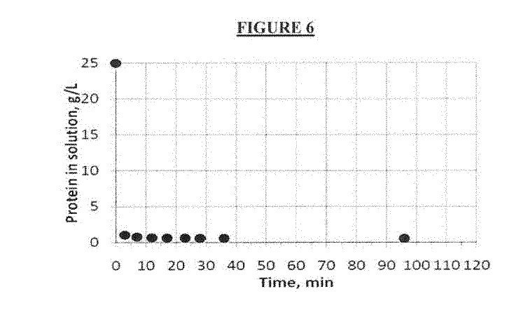

The kinetics of crystallization from a 6 ml stirred batch and a 1 L stirred batch were compared. The 6 ml stirred batch was prepared at 10° C. with stirring at 250 rpm using the following crystallization conditions: 10 g/L mAb01, 10 mM histidine, 20 mM NaCl, 16 mM TRIS, pH 6.8. FIG. 5 illustrates the kinetics of this reaction. The yield was 86.6% after 35 minutes, with a yield of 93.1% at equilibrium (0.69 g/L). Ninety percent of the equilibrium concentration was reached after about 30 minutes. The 1 L stirred batch was prepared at 10° C. with stirring at 150 rpm using the following crystallization conditions: 25 g/L mAb01, 52 mM trehalose, 10 mM histidine, 15 mM TRIS, pH 6.8. FIG. 6 illustrates the kinetics of this reaction. The yield was 95.8% after three minutes, with a yield of 98.3% at equilibrium (0.42 g/L). Ninety percent of the equilibrium concentration was reached in less than three minutes.

G. Stability of Pure mAb01 Crystals

Long-term storage was simulated by storing mAb01 crystals for one month at 20° C. after removing the liquid by centrifugation. As a liquid control, 70 g/L mAb01 in 20 mM histidine (pH 5.0) was also stored. SEC analysis indicated 0.5% aggregates and 1.5% fragments in the liquid formulation. The test crystals had only 0.3% aggregates and 1.4% fragments. These tests indicate that mAb01 crystals are amenable to long-term storage (e.g., are stable).

H. Protein Content

High concentration of mAb01 could be achieved by centrifugation. For example, centrifugation at 5252 g for three minutes provided a crystal pellet containing 214 g/L mAb01. And centrifugation at 50377 g for three minutes provided a crystal pellet containing 315 g/L mAb01, which is significantly higher than the maximum possible concentration of a liquid formulation.

I. Crystal Size and Length During Crystallization

The effect of stirring speed and protein concentration were assessed. The maximum crystal length in a 6 ml stirred batch reactor at 200 rpm was 60 μm. The maximum crystal length in a 6 ml stirred batch reactor at 120 rpm was 120 μm. Thus, a slower stirring speed allows for the formation of longer crystals. Crystallization at different mAb01 concentrations in 10 mM histidine, 250 mM trehalose, and TRIS to adjust the pH to 6.8 led to different crystal lengths (see Table 1).

TABLE 1

| mAb01 concentration | Mean crystal length after |
|---|---|
| (g/L) | crystallization, μm |
| 15 | 43.6 |
| 30 | 33.8 |
| 60 | 24.8 |
| 80 | 15.7 |
| 134 | 10.8 |
| 149 | 6.0 |

### Example 2

Crystallization of Antibody from Cell Culture Supernatant

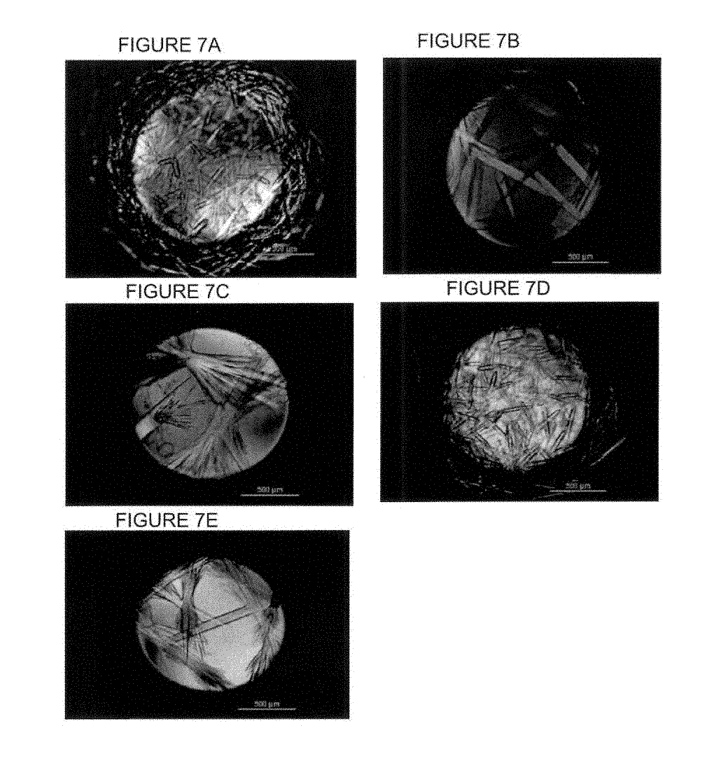

It was surprisingly found that mAb01 could be crystallized directly from cell-free culture supernatant. This supernatant was initially analyzed by SEC and found to contain many impurities. A 45 ml sample of mAb01-A cell culture supernatant (2.31 mg/ml mAb01) was concentrated to 4.5 ml by spin centrifugation (Amicon Ultra-15 Centrifugal Filter Unit with Ultracel-10 membrane). The concentrated supernatant was then dialyzed against 1 L 10 mM histidine buffer (pH 5) using a dialysis tubular membrane (Dialysis Tubing Visking (MWCO) 14000). The resulting 6.5 ml dialysate with a pH of 5.0 was clarified by centrifugation (5 min, 16100×g). This “pretreated harvest 1” had a mAb01 concentration of 12.9 g/L mAb01 (as measured by SEC) and conductivity of 0.7 mS cm⁻¹. Crystallization was then performed in μl batch experiments using Terasaki plates sealed with paraffin oil at 10° C. FIG. 7A shows mAb01 crystals prepared in a 10 μl batch consisting of 5 μl pre-treated harvest 1 and 5 μl crystallization solution 1 (12 mM TRIS, 20 mM NaCl) with a pH around 6.8 (confirmed from larger scale experiments). FIG. 7B shows mAb01 crystals prepared in a 10 μl batch consisting of 5 μl of solution (76.9 μl pre-treated harvest 1; 2.4 μl 0.2 M histidine buffer, pH 4.9; 45.7 μl water) and 5 μl crystallization solution 1 (12 mM TRIS, 20 mM NaCl). FIG. 7C shows mAb01 crystals prepared in a 10 μl batch consisting of 5 μl pre-treated harvest 1 and 5 μl crystallization solution (12 mM TRIS, 40 mM NaCl, pH 6.8). FIG. 7D shows mAb01 crystals prepared in a 10 μl batch consisting of 5 μl pre-treated harvest 1 and 5 μl crystallization solution (16 mM TRIS, 20 mM NaCl). FIG. 7E shows mAb01 crystals prepared in a 10 μl batch consisting of 5 μl of a solution (76.9 μl pretreated harvest 1, 2.4 μl 0.2 M histidine buffer, pH 4.9, and 45.7 μl water) and 5 μl crystallization solution 1 or crystallization solution 2, respectively. pH was 6.8. Each of the tested conditions provided compact crystals, thereby demonstrating that crystallization from batch concentrated, dialyzed cell culture supernatant was possible.

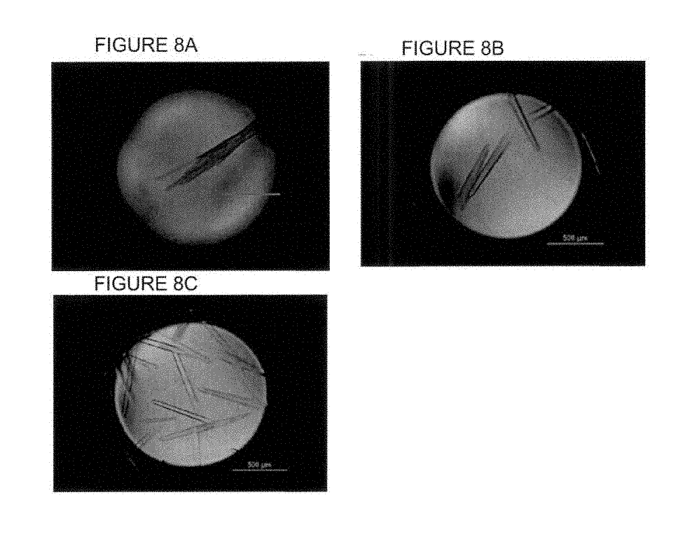

Experiments were also performed to determine if crystallization was possible without prior concentration. To this end, a 45 ml sample of mAb01-A cell culture supernatant (2.31 mg/ml mAb01) was dialyzed overnight against 5 L 10 mM histidine buffer (pH 5) using a dialysis tubular membrane (Dialysis Tubing Visking (MWCO) 14000). The resulting dialysate was clarified by centrifugation at 5252 rcf for 15 minutes followed by filtration using a 0.2 μm filter to produce “pretreated harvest 2”. Pretreated harvest 2 had a pH of 5.0 and a conductivity of 0.7 mS cm⁻¹. Twenty-five ml of the pre-treated harvest 2 was then dialyzed against 2.5 L 10 mM histidine buffer (pH 5) overnight. The resulting dialysate was centrifuged at 5252 rcf for 15 minutes and filtered using a 0.2 μm filter. This “pretreated harvest 3” had pH of 4.9 and conductivity of 0.6 mS cm⁻¹. Crystallization was then performed for pretreated harvest 2 and 3 (separately) in μl batch experiments using Terasaki plates using the following conditions (pH around 6.8): 5 μl pretreated harvest 2 and 5 μl containing 14 mM TRIS (FIG. 8A); 5 μl pretreated harvest 2 and 5 μl containing 12 mM TRIS; 5 μl pretreated harvest 2 and 5 μl containing 16 mM TRIS; 5 μl pretreated harvest 2 and 5 μl containing 16 mM TRIS and 20 mM NaCl; 5 μl pretreated harvest 2 and 5 μl containing 12 mM TRIS and 20 mM NaCl; 5 μl pretreated harvest 2 and 5 μl containing 12 mM TRIS and 40 mM NaCl; 5 μl pretreated harvest 2 and 5 μl containing 16 mM TRIS and 40 mM NaCl; 5 μl pretreated harvest 2 and 5 μl containing 14 mM TRIS and 4% PEG 10000; 5 μl pretreated harvest 2 and 5 μl containing 16 mM TRIS and 4% PEG 10000; 5 μl pretreated harvest 3 and 5 μl containing 10 mM TRIS; 5 μl pretreated harvest 3 and 5 μl containing 12 mM TRIS; 5 μl pretreated harvest 3 and 5 μl containing 14 mM TRIS (FIG. 8B); 5 μl pretreated harvest 3 and 5 μl containing 16 mM TRIS (FIG. 8C); 5 μl pretreated harvest 3 and 5 μl containing 12 mM TRIS, 20 mM NaCl; 5 μl pretreated harvest 3 and 5 μl containing 14 mM TRIS, 20 mM NaCl; 5 μl pretreated harvest 3 and 5 μl containing 14 mM TRIS, 40 mM NaCl; 5 μl pretreated harvest 3 and 5 μl containing 12 mM TRIS, 4% PEG 10000; 5 μl pretreated harvest 3 and 5 μl containing 14 mM TRIS, 4% PEG 10000; and, 5 μl pretreated harvest 3 and 5 μl containing 16 mM TRIS, 4% PEG 10000. Each of the tested conditions provided crystals, thereby demonstrating that crystallization from dialyzed cell culture supernatant, without batch concentration, was possible.

Crystallization from a 5 ml stirred batch was also tested. Water (2940 μl), 5M NaCl (10 μl), and pretreated harvest 1 were combined and mixed at 250 rpm, 10° C. Crystallization was initiated by adding 30 μl 1M TRIS to adjust the pH to around 6.8. The first crystals appeared after about 15 minutes, and the experiment was stopped after three hours. The crystals were separated by centrifugation (3 min, 16100 g), and dissolved in 0.5 ml 10 mM histidine buffer (pH 5). The pH was adjusted to 5 by adding 5 μl 10% acetic acid, resulting in about 650 μl solution containing mAb01. SEC analysis showed a high purity of 96.5%. The protein concentration of the dissolved crystal solution was 38.9 g/L mAb01.

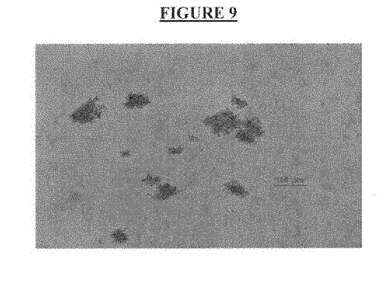

The effect of PEG on crystallization of a stirred batch (5 ml, 250 rpm, 10° C.) was also tested. A mixture of 2500 μl pretreated harvest 3, 2640 μl water, and 45 μl 1M TRIS was prepared and the pH adjusted to 6.8 by adding 18.5 μl 0.5 M acetic acid. The conductivity of this pretreated harvest 3 (without PEG) was 0.40 mS/cm⁻¹. The resulting crystals are shown in FIG. 9. SEC analysis showed a purity of 90.5%. Another stirred batch was prepared using 2500 μl pretreated harvest 3, 2210 μl water, 40 μl 1M TRIS, and 250 μl 40% PEG1000, and the pH adjusted to 6.8 by adding 2.5 μl 1M TRIS. The resulting conductivity of this pretreated harvest 3 PEG⁺ solution was 0.46 mS/cm⁻¹. It was determined that the addition of PEG or trehalose may increase the rate of nucleation but such substances are not necessarily required.

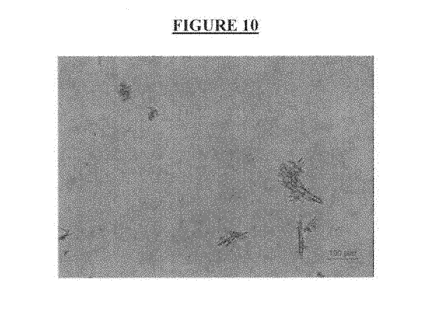

Pretreatment harvest 2 in a stirred batch was similarly tested. A stirred batch was prepared using 2500 μl pretreated harvest 2, 2215 μl water, 35 μl 1M TRIS, and 250 μl 40% PEG1000, and the pH adjusted to 6.8 by adding 8.0 μl 1M TRIS. The conductivity of this pretreated harvest 2 solution was 0.46 mS cm⁻¹. The resulting crystals are shown in FIG. 10. SEC analysis showed a purity of 92%. Only 0.3 g L⁻¹ antibody remained in the supernatant. This data demonstrates that very little antibody remained in the supernatant after crystallization and that the crystals contained high-purity antibodies. These experiments demonstrate that crystallization of mAb01 from a dialyzed harvest (pretreated harvest 2 or 3) in a 5 ml stirred batch is possible.

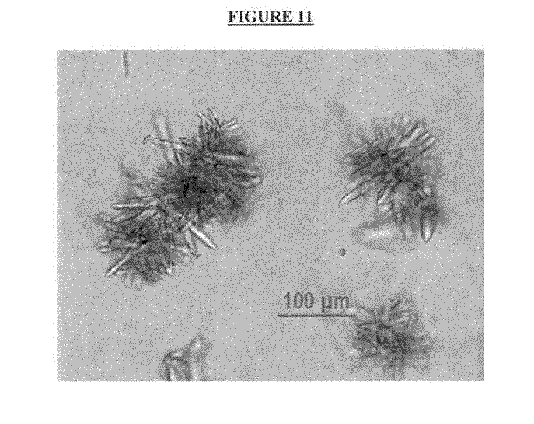

Crystallization from a 100 ml batch which was diafiltrated but not concentrated was also tested. A 100 ml cell-free harvest (mAb01-B, 3.3 g/L) was diafiltrated in a stirred reactor at 150 rpm (10° C.) against 400 ml 10 mM histidine, 10 mM NaCl, adjusted to pH 5.0 using acetic acid) using a crossflow ultrafiltration unit. Centrifugation was performed at 3200 rcf for 15 minutes followed by filtration through a 0.2 μm filter. The conductivity of this harvest (“pretreated harvest 4”) was 1.7 mS cm⁻¹. Sixty ml of pretreated harvest 4 was then crystallized in a 100 ml stirred batch reactor at 150 rpm (10° C.) by adding 2% w/v PEG 10000 and adjusting the pH to 6.8 by adding 0.7 ml 1M TRIS. Separation of the resulting crystals (discrete robust crystal rods) was accomplished by centrifugation for 15 min at 3200 rcf. SEC analysis showed a purity of 92%. These experiments showed that crystallization of high purity crystals from a diafiltrated harvest in a 100 ml stirred batch reactor was possible without a change of crystal morphology (e.g., FIG. 11).

mAb01 was also crystallized from a pretreated harvest by pH titration and diafiltration without prior concentration. A 752 ml cell-free harvest (mAb01-11506A, 3.3 g/L) was titrated to pH 5 using 10% acetic acid. The resulting precipitate was removed by centrifugation at 3200 rcf for 15 minutes. The supernatant was diafiltrated against 7 L of a 10 mM histidine buffer (adjusted to pH 5 with acetic acid) using a crossflow ultrafiltration unit (Sartorius stedim; MWCO 30 kDa; 30514459 02 E-SW Hydro-30K 004). During diafiltration, the supernatant was diluted to 994 ml with histidine buffer. The resulting precipitate was removed by centrifugation at 3200 rcf for 15 minutes followed by filtration using a 0.2 μm filter. The conductivity of this solution (pretreated harvest 5) was 0.7 mS cm⁻¹. Crystallization from pretreated harvest 5 was performed in a 1 L stirred batch reactor at 150 rpm (10° C.). Initially, 0.584 g NaCl and 19.88 g PEG 10000 were dissolved in pretreated harvest 5. Crystallization was initiated by adjusting the pH to 6.8 by adding 14 ml of 1 M TRIS. The first crystals were visible after one hour and crystallization completed by two hours. The crystals were separated by centrifugation (3200 rcf, 20 minutes) and dissolved in 10 mM histidine buffer.

The results of this process are summarized in Table 2. Afterwards, the antibody was recrystallized by adjusting the pH to 6.8. For analysis, the recrystallized antibody crystals were separated by centrifugation (3200 rcf, 20 minutes) and dissolved in 10 mM histidine buffer. These experiments demonstrate that crystallization from a pH titrated and diafiltrated harvest was possible. Scale-up into a 1 L stirred batch reactor was successful. Crystallization was surprisingly fast. The total process demonstrated a high yield (75%) (Table 2). And successful purification was confirmed by SEC and host cell protein (HCP) analysis, which is comparable to purification by Protein A chromatography.

TABLE 2

| | Yield of | HCP, | Purity by |
|---|---|---|---|
| Probe | step (%) | ppm | SEC, % |
| Initial harvest | — | 81752 | — |
| Pretreated harvest 5 | 87 | — | — |
| Dissolved crystals | 88 | 11259 | 92.9 |
| Dissolved crystals after | 98 | 4733 | 98.5 |
| recrystallization | | | |

| | |
|---|---|
| Total yield (%) | 75 |

A stability test at 20° C. was also performed. Following crystallization, crystals were separated by centrifugation for three minutes at 44,000 rcf and the supernatant removed. mAb01 crystals (about 220 g/L) were stored and compared to a liquid control sample (70 g·L mAb01, 20 mM histidine, pH 5.0). The results are shown below in Table 3:

TABLE 3

| | Aggregates, % | Fragments, % |
|---|---|---|
| 20° C. control | 0.5 | 1.5 |
| 20° C. crystals | 0.3 | 1.4 |

The results showed no disadvantage of a crystalline formulation compared to the liquid control after one month.

Crystallization of mAb01 from a diluted cell-free supernatant harvest in the presence of pure mAb01 was also performed in a μl batch (10° C., 10 mM histinde, 10 mM TRIS, PEG 10000; pure mAb01, and mAb01 harvest (with 3.3 g/L mAb01)). Crystallization was possible using the conditions, as shown in Table 4.

TABLE 4

| PEG 10000 | Added pure mAb01 | Added harvest |
|---|---|---|
| (% w/v) | (g/L) | (% v/v) |
| 0 | 5; 10 | 15-25 |
| 1 | 10 | 40 |
| 1 | 5 | 30 |
| 1 | 2 | 15-20 |
| 2 | 5 | 40 |
| 2-5 | 10 | 35-40 |
| 3-5 | 5 | 35-40 |
| 2 | 2 | 30-50 |
| 3 | 2 | 35-45 |
| 4; 5 | 2 | 30-40 |

This data showed that up to 50% harvest was tolerated in the crystallization process. It can be seen that: 1) PEG 10000 reduced the effect of inhibiting salts in the cell-free supernatant (e.g., harvest); 2) crystallization of mAb01 from diluted mAb01 harvest including pure mAb01 is possible; 3) crystallization of mAb01 from harvest without precipitation is feasible. Thus, it is possible to crystallize mAb01 by concentrating the cell-free supernatant (e.g., harvest) without precipitation, dilute the cell-free supernatant without precipitation, and subsequently crystallize mAb01.

Crystallization of mAb01 by Concentrating and Diluting Cell-Free Harvest

Cell-free harvest containing 3.2 g/L mAb01 was concentrated by a factor between 4 and 10. Afterwards the concentrated harvest was diluted with a buffer suitable for crystallization and the resulting solution was crystallized in a stirred mL reactor at 250 rpm at 10° C. by adjusting the pH around 6.8. After the crystallization, the crystals were separated by centrifugation and analyzed by SEC (see Table 5).

TABLE 5

| | Dilution | | | | |
|---|---|---|---|---|---|
| Concentration | factor of the | | | | |
| factor of | concentrated | Crystallisation | Crystallization | Yield | Purity by |
| harvest | harvest | conditions | volume, mL | (%) | SEC (%) |
| 5 | 2.5 | 40% (v/v) concentrated | 6 | 53 | 92 |
| | | harvest, 10 mM histidine, | | | |
| | | 12 mM TRIS, 2% | | | |
| | | PEG10000, acetic acid to | | | |
| | | adjust the pH to 6.8 | | | |
| 5 | 2.5 | 40% (v/v) concentrated | 6 | 46 | 81 |
| | | harvest, 10 mM histidine, | | | |
| | | 2% PEG10000, acetic | | | |
| | | acid to adjust the pH to 6.8 | | | |
| 4 | 2.5 | 40% (v/v) concentrated | 8 | 57 | 92 |
| | | harvest, 10 mM histidine, | | | |
| | | 2% PEG10000, acetic | | | |
| | | acid to adjust the pH to 6.8 | | | |
| 10 | 3.3 | 30% (v/v) concentrated | 6 | 58 | 93 |
| | | harvest, 10 mM histidine, | | | |
| | | 2% PEG10000, acetic | | | |
| | | acid to adjust the pH to 6.8 | | | |
| 10 | 3.3 | 30% (v/v) concentrated | 6 | 66 | 87 |
| | | harvest, 10 mM histidine, | | | |
| | | acetic acid to adjust the | | | |
| | | pH to 6.8 | | | |
| 10 | 3.3 | 30% (v/v) concentrated | 6 | 65 | 94 |
| | | harvest, 10 mM histidine, | | | |
| | | 1% PEG10000, acetic | | | |
| | | acid to adjust the pH to 6.8 | | | |

Crystallization of mAb01 from Partly Purified Solutions

mAb01 from harvest was first partly purified in a traditional way (Protein A chromatography) was performed, followed by a virus inactivation at low pH (this solution was called VIN). Afterwards, purification by anion exchange chromatography was performed (this solution was called AEC). mAb01 from VIN and AEC was crystallized in a stirred 6 mL crystallizer at 8 g/L mAb01 by adding histidine to 10 mM and adjusting the pH to about 6.8 by adding several μL of 1 M Tris. After the first crystallization, the crystals were either dissolved and recrystallized or washed in 10 mM histidine buffer pH 6.8. The yield, the purity, the HCP content and the biological activity were quantified (see Table 6).

TABLE 6

| | Yield of | Purity | HCP, | Biological |
|---|---|---|---|---|
| Probe | the step, % | (SEC), % | ppm | activity, % |
| VIN | | 98.8 | 2656 | 89.3 |
| VIN crystallized | 94.4 | 98.8 | 1935 | 93.8 |
| VIN recrystallized | 96.8 | 99.0 | 1290 | 96.4 |
| VIN washed | 97.0 | 98.8 | 1489 | 95.0 |
| AEC | | 99.1 | 29 | 88.7 |
| AEC crystallized | 93.1 | 99.2 | 8 | 93.0 |
| AEC recrystallized | 95.5 | 99.2 | 5 | 91.9 |
| AEC washed | 96.5 | 99.2 | 7 | 90.9 |

The SEC analysis showed that no aggregation or degradation occurred as a result of the crystallization process and that a high level of purification was achieved. The bioassay showed that biologically active protein was preferably incorporated into the crystals. A clear HCP reduction was visible in all crystallization and washing steps. Starting from the AEC step, crystallization reached the same HCP reduction compared to CEC.

Suitability of Crystallization in an Existing Large-Scale GMP Purification Process

A scaled-up purification process in a one-liter scale was tested. The purification consisted of: pretreatment of the harvest, crystallization, recrystallization, virus inactivation at low pH, anion exchange chromatography, nanofiltration, and final crystallization. The starting material was cell-free harvest. The 1.2 L cell-free harvest was concentrated by factor 6 using a 10 kDa MW cut-off membrane (Sartocon® Slice). Afterwards, the pH was titrated to pH 5.0 by adding 10 mL 1.2 M acetic acid, and the solution was clarified by centrifugation (15 min, 3200 rcf). Using the same membrane, the buffer was exchanged by five diafiltration volumes (10 mM histidine buffer, pH 5.0 adjusted with acetic acid). The solution was clarified by centrifugation (15 min, 3200 rcf) and filtration (0.2 μm). This pretreatment process had a yield of 94.7%. The solution was diluted with 10 mM histidine buffer, pH 5.0 (adjusted with acetic acid) to one liter total volume. The conductivity was 0.5 mS cm⁻¹. The crystallization was performed in a stirred one liter reactor at 10° C. at 150 rpm. Crystallization conditions were adjusted by adding 0.876 g sodium chloride and 13 mL 1M TRIS (led to a conductivity of 1.8 mS cm⁻¹ and a pH of 6.77). Additionally, 2% w/v PEG 10000 were added. Crystals were separated by centrifugation (15 min, 3200 rcf) and dissolved in 10 mM histidine buffer pH 5 resulting in 116 ml of a solution with a conductivity of 0.8 mS cm⁻¹ and a pH of 5.2. The yield of the crystallization was 87.2%. A recrystallization was performed in a 100 mL scale stirred crystallizer at 10° C. and 200 rpm. Crystallization was started by addition of 0.112 g sodium chloride and 1.9 mL 1M TRIS (which led to a conductivity of 2.0 mS cm⁻¹ and a pH of 6.8). Crystals were separated as before. Afterwards, a standard virus inactivation step at low pH, an anion exchange chromatography step, and a nanofiltration step were accomplished easily after the crystallization without encountering any problems. Hence, it was shown that the proposed process can be operated under GMP requirements. A final crystallization in the presence of 250 mM trehalose was performed to achieve an isotonic solution, which is important for injectable suspensions. The total process led to a 3030 fold HCP reduction. Surprisingly, no DNA was present any more already after the recrystallization step (see Table 7).

TABLE 7

| | Purity | DNA, | HCP, |
|---|---|---|---|
| Step | (SEC), % | ppb | ppm |
| Cell-free harvest | | about | 266719 |
| | | 77664 | |
| pH 5 titration | 86 | | 214209 |
| and clarification | | | |
| Diafiltration and | 92 | | 222830 |
| clarification | | | |
| Crystallization | 97 | | 39070 |
| Recrystallization | 97 | <2 | 17354 |
| Virus | 98 | | 13864 |
| inactivation | | | |
| Anion exchange | 99 | | 1506 |
| chromatography | | | |
| Nanofiltration | 99 | | 1289 |
| Final | 99 | <2 | 88 |
| crystallization | | | |

While the present invention has been described in terms of the preferred embodiments, it is understood that variations and modifications will occur to those skilled in the art. Therefore, it is intended that the appended claims cover all such equivalent variations that come within the scope of the invention as claimed.

What is claimed is:

1. A method for preparing purified monoclonal antibodies, the method comprising:
a) introducing a low ionic strength buffer into a composition comprising monoclonal antibodies, wherein impurities precipitate from the composition, and wherein the pH of the low ionic strength buffer is at a pH where the antibody is soluble and does not crystallize or precipitate;
b) removing the precipitate to produce a first clarified composition;
c) optionally introducing a low ionic strength buffer into the clarified composition, wherein impurities precipitate from the composition to produce a second clarified composition, and wherein the pH of the low ionic strength buffer is at a pH where the antibody is soluble and does not crystallize or precipitate;
d) removing the precipitate from the composition of step c);
e) adjusting the pH of the first or the second clarified composition to about the pI of the monoclonal antibody and optionally introducing one or more additives to produce crystals; and,
f) isolating the crystals formed in step e).

2. The method according to claim 1, wherein the composition comprising monoclonal antibodies is a cell-free cell culture supernatant comprising monoclonal antibody, and wherein steps a) and c) include dialyzing the cell cell-free culture supernatant or the clarified supernatant, respectively, against the low ionic strength buffer, and wherein between steps a) and b) and/or between steps b) and c) the supernatant may optionally be concentrated.

3. The method of claim 1, wherein the low ionic strength buffer provides a conductivity of less than or equal to 12 mS cm⁻¹.

4. The method of claim 3, wherein the low ionic strength buffer provides a conductivity of 4 mS cm⁻¹ or less.

5. The method of claim 4, wherein the low ionic strength buffer provides a conductivity of 2 mS cm⁻¹ or less.

6. The method of claim 1, wherein the low ionic strength buffer
(i) is a histidine buffer,
(ii) comprises at least one or more salts, and/or
(iii) comprises at least one or more sugars.

7. The method of claim 1, wherein the pH is adjusted using
(i) a Tris buffer, and/or
(ii) a buffer comprising one or more additives selected from the group consisting of sodium chloride, polyethylene glycol, and a sugar.

8. The method of claim 1, wherein at least about 50% of the antibody contained in the cell-free culture supernatant is isolated in the isolating step.

9. The method of claim 1, wherein the purity of the crystallized antibody is at least about 90%.

10. The method of claim 1, further comprising
(i) dissolving the isolated crystals in a solution,
(ii) re-crystallizing the monoclonal antibody by adjusting the pH of the solution to about the pI of the monoclonal antibody, and/or
(iii) controlling crystal size by adjusting the starting protein concentration of the cell culture supernatant.

11. The method of claim 1, further comprising controlling crystal size by stirring the substrate at a particular speed.

12. The method of claim 11, wherein crystallization occurs with stirring at a power input per volume of less than 1 W L⁻¹.

13. The method of claim 11, wherein the maximum local energy dissipation (εₘₐₓ) is between 0.009 W kg⁻¹ and 1.3 W kg⁻¹, in particular between 0.1 to 0.4 W kg⁻¹.

14. The method of claim 11, wherein a three-bladed segment impeller is used for stirring.

15. A method for preparing monoclonal antibodies in crystal form directly from cell culture supernatant, the method comprising:
a) dialyzing cell-free cell culture supernatant comprising monoclonal antibody against a low ionic strength buffer;
b) removing precipitate formed in step a) from the supernatant, if present therein, to produce a clarified supernatant;
c) optionally concentrating the clarified supernatant;
d) optionally dialyzing the clarified supernatant of b) or c) against a low ionic strength buffer to produce a pretreated solution;
e) removing precipitate from the pretreated solution of step d), if present therein;
f) adjusting the pH of the pretreated solution of step d) or e) to about the pI of the monoclonal antibody and optionally introducing one or more additives to produce crystals; and,
g) isolating the crystals formed in step f).

16. The method of claim 15, comprising concentrating the cell-free cell culture supernatant before step b).
